(19) United States
(12) Patent Application Publication
(10) Pub. No.: US 2015/0359420 A1
(43) Pub. Date: Dec. 17, 2015

(54) ENDOSCOPE AND METHOD OF MANUFACTURING ENDOSCOPE

(71) Applicant: Panasonic Intellectual Property Management Co., Ltd., Osaka (JP)

(72) Inventors: Yuichi Hatase, Fukuoka (JP); Haruhiko Kohno, Fukuoka (JP); Ken Shimonosono, Fukuoka (JP)

(21) Appl. No.: 14/728,857

(22) Filed: Jun. 2, 2015

(30) Foreign Application Priority Data
Jun. 11, 2014 (JP) 2014-120869

(51) Int. Cl. A61B 1/05; H05K 3/32; A61B 1/00

(52) U.S. Cl. CPC A61B 1/051; A61B 1/0011; A61B 1/00114; H05K 3/32

(57) ABSTRACT

An imaging unit at a tip end portion of an insertion section has a board on which the image pickup device is installed, a cable through which a signal is input to and output from the board, and a ground bar which is provided so as to protrude from the surface of the board and is connected to a ground of the board. In a distal portion of the cable on the imaging unit side, a shield part is connected to the ground of the board through the ground bar and a core line is directly assembled on the board. The ground bar supports the cable on the board such that the height of the cable in the vicinity of a board-mounting portion of the cable becomes greater than or equal to a predetermined value.

### BACKGROUND OF THE INVENTION

1. Field of the Invention

The present invention relates to a compact endoscope which performs observation, surgery, or the like on a constricted site in the medical field or the industrial field, and a method of manufacturing the endoscope.

2. Description of the Related Art

In the related art, an endoscope for imaging the inside of a body of a patient, a machine, or a structure has been widespread in the medical field or the industrial field. As this type of endoscope, a configuration has been known in which an image of light from an imaging site is formed on a light receiving surface of an image pickup device using an objective lens system in an insertion section which is inserted inside an observation object; and the imaged light is converted to an electrical signal which is then transmitted to an image processing apparatus or the like outside as a video signal through a signal cable.

A large number of components such as an image pickup device and an optical element, such as a lens, which forms an optical image on an imaging surface of the image pickup device, are disposed in a hard part which is provided at a tip end of this kind of endoscope. In addition, a configuration has been known in which the imaging direction, that is, the visual field is changed based on an operation of an operator or the like by making the hard part be connected to the insertion section which is bendable. In recent years, in the endoscope with such a complicated configuration, further reduction in outer diameter becomes important in order to more easily manufacture the endoscope and to reduce a burden of a person to be treated.

For example, in Japanese Patent Unexamined Publication No. 7-327916, a visual field direction change type endoscope is disclosed which has a visual field direction change unit that sets a visual field range by making a part of a light beam, which is incident from a transparent window disposed from a tip end surface of a tip end of an insertion section to the middle of a side portion in the vicinity of the tip end, selectively incident on an objective optical system of an imaging portion which is provided so as to freely rotate; and can freely switch the direction of the visual field of the endoscope in accordance with the use thereof and an observation object.

In the endoscope, there is a tendency that the size of the tip end portion of the insertion section is increased compared to a configuration in which the imaging portion is fixed, in the case of the visual field direction change type configuration in which the imaging portion that is installed with the above-described objective optical system and an image pickup device is made to freely rotate. In the example in the related art as disclosed in PTL 1, it is difficult to reduce the size of the insertion section since a large amount of space is required on a board in order to assemble a component, such as an image pickup device, and a signal line on the board which is provided in the imaging portion which is freely rotatable.

### SUMMARY OF THE INVENTION

The present invention has been made in consideration of the above circumstances, and an object of the present invention is to provide an endoscope enabling miniaturization of an insertion section at a tip end portion, and a method of manufacturing the endoscope.

According to an aspect of the present invention, there is provided an endoscope which has an imaging unit at a tip end portion of an insertion section. The imaging unit has a board on which the image pickup device is installed, a cable through which a signal is input to and output from the board, and a ground bar which is provided so as to protrude from the surface of the board and is connected to a ground of the board. In a distal portion of the cable on the imaging unit side, a shield part is connected to the ground of the board through the ground bar and a core line is directly assembled on the board.

According to another aspect of the present invention, there is provided an endoscope which has an imaging unit at a tip end portion of an insertion section. The imaging unit has a board on which the image pickup device is installed, an assembly section which is assembled on the board, and a cable through which a signal is input to and output from the board. The assembly section and the cable are assembled on the board using a plurality of types of conductive connection materials which have different melting points.

According to still another aspect of the present invention, there is provided a method of manufacturing an endoscope which includes an imaging unit at a tip end portion of an insertion section, the imaging unit having a board on which the image pickup device is installed, a cable through which a signal is input to and output from the board, and a ground bar which is provided so as to protrude from the surface of the board and is connected to a ground of the board, the method including assembling an assembly section and the cable to be assembled on the board on the board using a plurality of types of conductive connection materials which have different melting points, in a distal portion of the cable on the imaging unit side, when a shield part is connected to the ground of the board through the ground bar and a core line is directly assembled on the board.

According to the present invention, it is possible to achieve miniaturization of the insertion section at the tip end portion in the endoscope.

### BRIEF DESCRIPTION OF DRAWINGS

FIG. 1 is an overall configuration view of an endoscope according to the present embodiment;

FIG. 2 is an explanatory view showing a configuration of a connection part and a relationship between a state of the connection part and a curved state of a bending portion;

FIG. 3 is an explanatory view showing a configuration of the connection part and a relationship between a state of the connection part and a curved state of the bending portion;

FIG. 4 is an explanatory view showing a configuration of the connection part and a relationship between a state of the connection part and a tilted state of an imaging unit of a hard part;

FIG. 5 is an explanatory view showing a configuration of the connection part and a relationship between a state of the connection part and a tilted state of the imaging unit of the hard part;

FIG. 6A is a transparent view showing a configuration of the hard part which is attached to the bending portion on a free end side;

FIG. 6B is a transparent view showing a configuration of the hard part which is attached to the bending portion on the free end side;

FIG. 7 is a transparent view showing an arrangement configuration of the imaging unit in the hard part;

FIG. 8A is a side view showing a first configuration example of a connection portion between a board and a cable in an imaging unit;

FIG. 8B is a perspective view showing the first configuration example of the connection portion between the board and the cable in the imaging unit;

FIG. 9A is a side view showing a second configuration example of a connection portion between a board and a cable in an imaging unit;

FIG. 9B is a perspective view showing the second configuration example of the connection portion between the board and the cable in the imaging unit;

FIG. 10 is a perspective view showing a third configuration example of a connection portion between a board and a cable in an imaging unit;

FIG. 11 is a side view showing a fourth configuration example of a connection portion between a board and a cable in an imaging unit;

FIG. 12A is a transparent side view for illustrating an assembling possible area of a component in the board of the imaging unit of the present embodiment;

FIG. 12B is a plan view for illustrating the assembling possible area of the component in the board of the imaging unit of the present embodiment;

FIG. 12C is a transparent perspective view for illustrating the assembling possible area of the component in the board of the imaging unit of the present embodiment;

FIG. 13A is a transparent side view for illustrating an assembling possible area of a component in a board of an imaging unit of a comparative example;

FIG. 13B is a plan view for illustrating the assembling possible area of the component in the board of the imaging unit of the comparative example;

FIG. 14A is a perspective view showing a first example of an assembling procedure of a cable on a board; and

FIG. 14B is a perspective view showing a second example of an assembling procedure of a cable on a board.

### DETAILED DESCRIPTION OF THE PREFERRED EMBODIMENT

Hereinafter, the embodiment according to the present invention will be described with reference to accompanying drawings. The directions used in the description are basically set to comply with a description of a direction in each drawing. However, in some cases, the direction in which a section formed in a cylindrical shape or a bar shape extends is called an “axial direction” and the direction of a rotary axis of a rotating section is called the “axial direction”. In addition, in some cases, the direction facing the inside and the outside of an axis around the axis is called a “radial direction” and the direction of rotating around an axis is called a “peripheral direction”. In addition, in some cases, when a cross section of a section which is orthogonal to an axial direction has a rectangular shape, the direction thereof is called the “radial direction” and the “peripheral direction” for convenience.

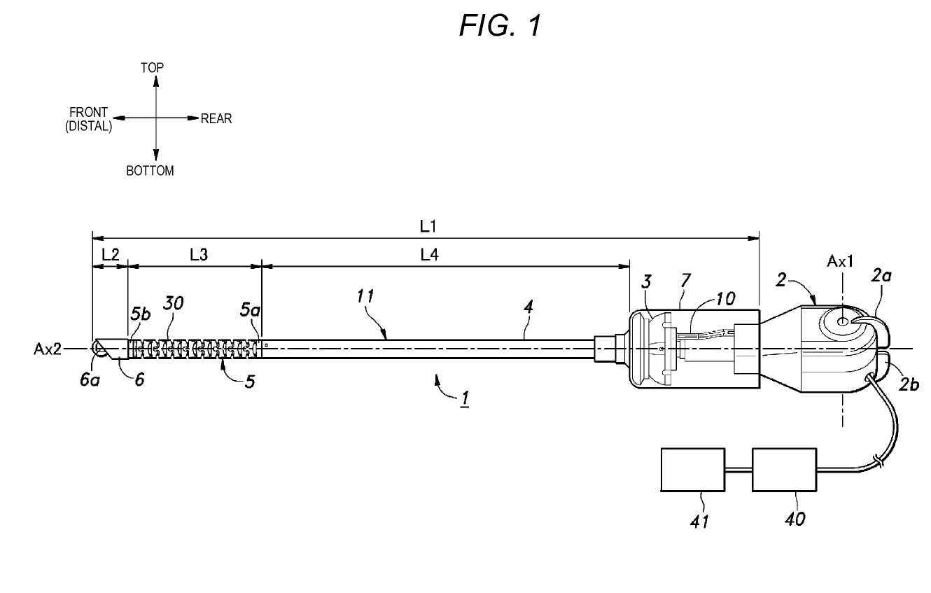

FIG. 1 is an overall configuration view of endoscope 1 according to the present embodiment. An endoscope which is used in surgery of the abdomen of a human in the medical field is exemplified in the present embodiment, and the configuration thereof is exemplified.

Endoscope 1 mainly has grip part 2, connection part 3, and insertion section 11 which is inserted into a site to be observed. Insertion section 11 has linear pipe-like straight portion 4 which is connected to grip part 2 from a proximal side through connection part 3 and cannot be curved; bending portion 5 which is configured so as to be able to be curved; and hard part 6 at a tip end portion in which imaging unit 6a is accommodated as an example of a functional section. Rotating operation part 7 which is rotated in an extending direction of straight portion 4 as an axis is provided in an outer circumferential portion of connection part 3.

As the dimensions of each part of endoscope 1, for example, length L1 from a tip end of hard part 6 to a rear end of rotating operation part 7 is about 600 mm, length L2 of hard part 6 is about 15 mm, length L3 of bending portion 5 is about 50 mm, and length L4 of straight portion 4 is about 450 mm. In addition, the outer diameters of hard part 6, bending portion 5, and straight portion 4 are set to about 10 mm at maximum.

When performing laparoscopic surgery using endoscope 1, hard part 6 and bending portion 5 of insertion section 11 are guided to a surgical site through a trocar or a trocar tube. Meanwhile, a part of straight portion 4 of insertion section 11 on a proximal side enters a state of being exposed to the outside of the body and the surgery is performed while an operator or the like grips and operates grip part 2.

Grip part 2 is provided with first operation part 2a which is for performing an operation such that bending portion 5 is curved, and a second operation part 2b for operating the direction of imaging using imaging unit 6a which is installed in hard part 6. When an operator or the like operates first operation part 2a, bending portion 5 is curved toward a predetermined direction (for example, downward in the drawing) in accordance with the operation amount and the imaging direction of imaging unit 6a which is provided in hard part 6 changes, that is, the visual field of the imaging unit moves. In grip part 2, first operation part 2a is rotatable around first axis Ax1 which is a rotary axis of the operation part. At this time, the rotating direction of first operation part 2a and the curving direction of bending portion 5 are configured so as to coincide with each other in consideration of operability.

In the following description, an operation through which a visual field is moved by curving of bending portion 5 by operating first operation part 2a is called a “curving operation”; an angle which is formed by a direction to which a tip end of hard part 6 faces through the curvature and a central axis direction (second axis Ax2) of straight portion 4 is called a “curving angle”; and a direction to which the tip end of hard part 6 faces through the curvature in a front view is called a “curving direction”. In some cases, for example, curving of bending portion 5 such that the tip end of hard part 6 faces downward (upward) in the drawing is expressed as “curving downward (upward).

In addition, in grip part 2, second operation part 2b is also rotatable around first axis Ax1. When an operator or the like operates second operation part 2b, imaging unit 6a which is pivotally supported in hard part 6 rotates and the imaging direction of imaging unit 6a changes, that is, the visual field moves. Here, the visual field of imaging unit 6a moves between a forward direction and a downward direction in the drawing.

In the following description, an operation through which a visual field is moved by operating second operation part 2b is called a “tilting operation” or simply called “tilt”. The operation range (rotation range around first axis Ax1 as an axis) of first operation part 2a and second operation part 2b is restricted by a stopper (not shown) which is provided in grip part 2. First operation part 2a and second operation part 2b may have a configuration using a rotational grip or the like in addition to the lever type configuration shown in the drawing.

FIG. 1 shows an initial state of endoscope 1. Bending portion 5 at this time is in a linear shape and the visual field of imaging unit 6a in hard part 6 faces forward in the drawing. When first operation part 2a is operated from this state, bending portion 5 is curved downward in the drawing. When second operation part 2b is operated, imaging unit 6a is tilted downward in the drawing. For example, when the curving angle of bending portion 5 is set to 0° to 90° and the tilt angle of imaging unit 6a is set to 0° to 90°, it is possible to enlarge the movement range of the visual field within a range of 0° to 180° without increasing the curving angle of bending portion 5 by combining the curving operation and the tilting operation (that is, without occupying a large amount of space during curving). In endoscope 1 of the present embodiment, the direction (imaging direction), to which a tip end of a functional section faces through curving of bending portion 5 which was in a linear shape in an initial state, and the direction, to which the tip end of the functional section faces through rotating of the functional section itself which was pivotally supported in hard part 6, substantially coincide with each other, and therefore, the movement angles of the visual field are summed by the combination of the curving operation and the tilting operation.

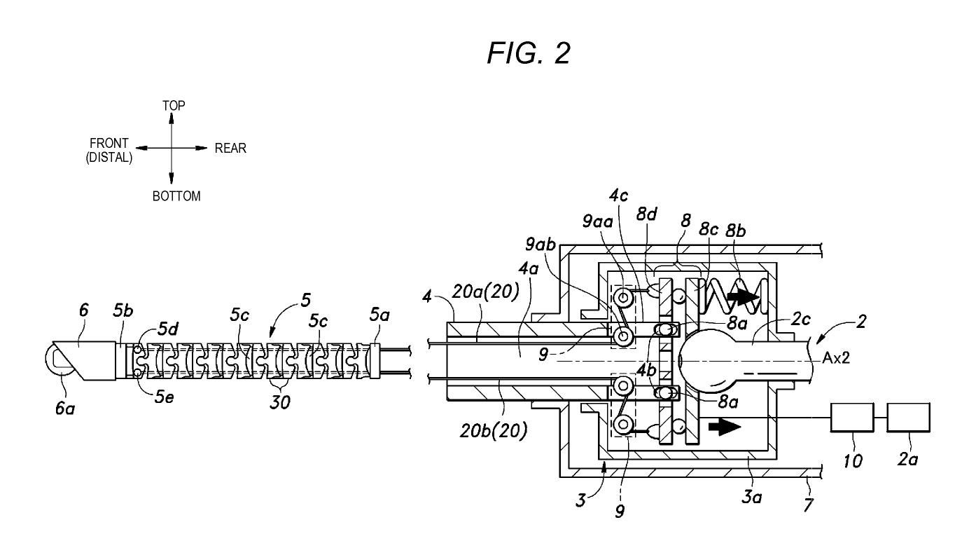

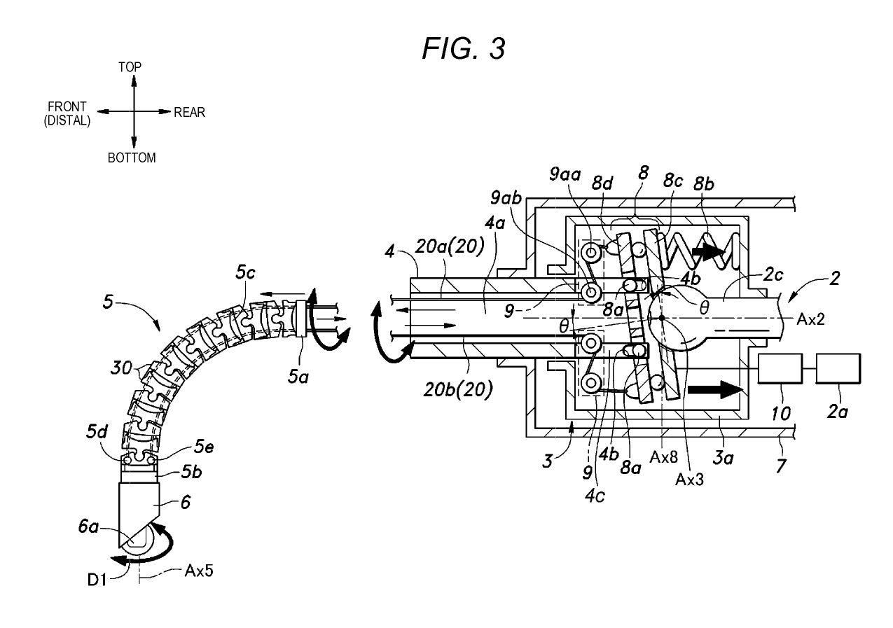

FIGS. 2 and 3 are explanatory views showing a configuration of connection part 3 and a relationship between a state of connection part 3 and a curved state of bending portion 5. In straight portion 4 and connection part 3 of insertion section 11 on a proximal side, a partial cross section of an internal configuration relating to a curving operation is shown in FIGS. 2 and 3. FIG. 2 shows a state (initial state) in which bending portion 5 is in a linear shape and FIG. 3 shows a state in which bending portion 5 is curved downward in the drawing.

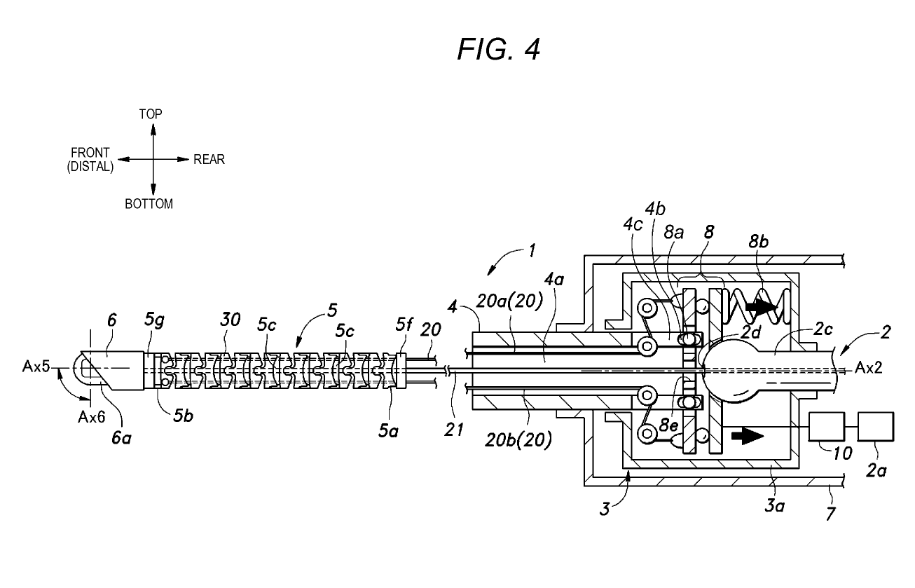

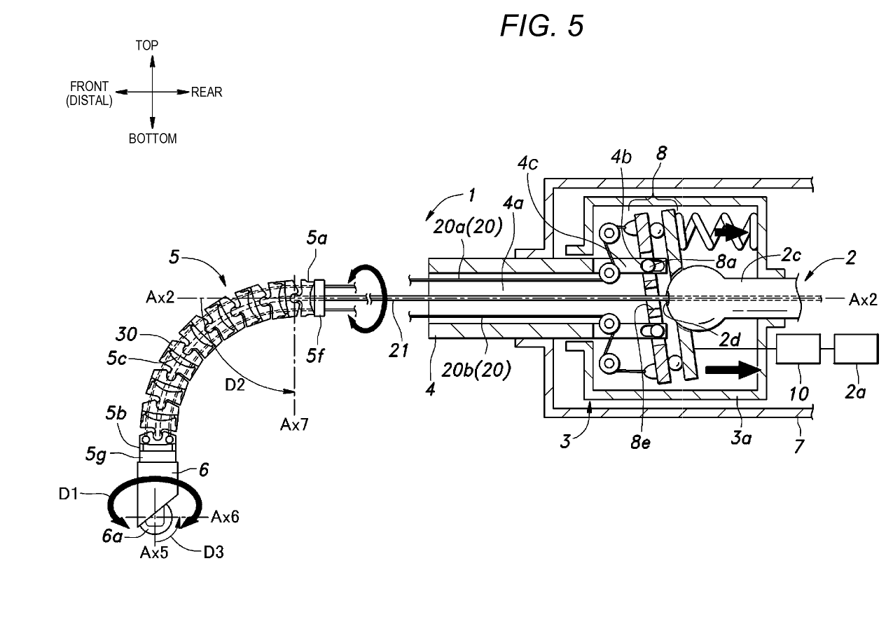

In addition, FIGS. 4 and 5 are explanatory views showing a configuration of connection part 3 and a relationship between a state of connection part 3 and a tilted state of imaging unit 6a of hard part 6. Similarly to FIGS. 2 and 3, in straight portion 4 and connection part 3 of insertion section 11 on a proximal side, a partial cross section of an internal configuration relating to a tilting operation is shown in FIGS. 4 and 5. FIG. 4 shows a state (initial state) in which bending portion 5 is in a linear shape and FIG. 5 shows a state in which bending portion 5 is curved downward in the drawing.

Connection part 3 on a proximal side (rear side in the drawing) is supported by grip part 2 and is connected to straight portion 4 on a front side in the drawing. In addition, connection part 3 and grip part 2 are connected to each other through ring section 10 (refer to FIG. 1 or the like). Force (hereinafter, force generated through an operation of first operation part 2a or second operation part 2b will be referred to as “operating force”) generated by operating first operation part 2a is transmitted to connection part 3 by link section 10. Operation wire 20, which is a first power transmission section is inserted into the insertion section starting from connection part 3 to straight portion 4 and bending portion 5, and is provided so as to be movable within the insertion section. As will be described later, in connection part 3, the operating force of first operation part 2a is converted to traction force of operation wire 20 and the traction force is transmitted to free end 5b of bending portion 5 on a tip end side.

Straight portion 4 is a cylindrical or linear section, which has hollow portion 4a extending in a second axis Ax2 direction, and is formed of, for example, stainless steel. Proximal end 5a of bending portion 5 is attached to one end of straight portion 4 on a tip end side. The other end of the straight portion on a proximal side is connected to grip part 2 through connection part 3 and extends forward from grip part 2.

Torque tube 21 (refer to FIGS. 4 and 5), which is a second power transmission section, is inserted into the insertion section starting from grip part 2 to straight portion 4 and bending portion 5 through connection part 3 and is provided so as to be rotatable. The operating force generated through an operation of second operation part 2b is converted to rotating force of torque tube 21, which has second axis Ax2 as an axis, by a gear mechanism which is provided inside grip part 2, and the rotating force is transmitted to hard part 6 on the tip end of the insertion section. Sphere bearing 2c which is engaged with connection part 3 is provided in grip part 2 so as to protrude forward in the drawing. Bearing opening 2d which penetrates in the second axis Ax2 direction is provided at the center of sphere bearing 2c. The rotating force of torque tube 21 is directly transmitted to hard part 6 without passing through connection part 3.

In addition, endoscope 1 is connected to video processor 40 and display apparatus 41 through a cable from grip part 2. An image signal of a still image or a moving image, which is obtained by imaging an observation object (here, the inside of a human body) using imaging unit 6a of hard part 6, is transmitted to video processor 40, and various signal processing or the like is performed in video processor 40. The image to be observed which has been processed by video processor 40 is displayed on display apparatus 41. In contrast, endoscope 1 is operated by receiving electric power and various control signals from video processor 40, and imaging is performed in imaging unit 6a at a timing based on the control signals.

First, the configuration relating to the curving operation of bending portion 5 will be described with reference to FIGS. 1, 2, and 3. Here, an example of a basic configuration in which bending portion 5 is made to be able to be curved in one direction (vertical direction in the drawing) is shown.

Bending portion 5 extends from proximal end 5a to free end 5b and is configured to have a plurality of joint pieces 30 which are connected between proximal end 5a and free end 5b. In the following description, in some cases, the axis constituted by an aggregate of the plurality of joint pieces 30 is called an “axis of bending portion 5” and the direction thereof is called an “axial direction of bending portion 5”. Since bending portion 5 can be curved, the “axial direction of bending portion 5” changes in accordance with the curving direction and the curving angle.

Joint piece 30 is, for example, formed of stainless steel and is a section which forms a rectangular shape with round corners or a circular shape when viewed from the axial direction of bending portion 5. The joint piece is configured such that free end 5b of bending portion 5 is curved in an arbitrary direction with respect to proximal end 5a by alternately shifting the plurality of joint pieces 30 by 90° to connect them to each other. In addition, operation wires 20 (first operation wire 20a and second operation wire 20b) are inserted into the inside of the plurality of joint pieces 30, and bending portion 5 is curved by pulling one of operation wires 20 and relaxing the other.

As shown in FIGS. 2 and 3, connection part 3 is configured to have connection part housing 3a, and traction section 8 and wire guide 9 which are provided in connection part housing 3a. Connection part housing 3a of connection part 3 is engaged with sphere bearing 2c, which protrudes forward in the drawing from grip part 2, on the rear side of the connection part housing and is fixed in a state where the rotation around second axis Ax2 as an axis and the movement in a frontward-rearward direction are restricted. In addition, connection part housing 3a freely rotatably supports straight portion 4 in the front part around second axis Ax2 as an axis. The central axis of straight portion 4 is supported by connection part housing 3a so as to coincide with the central axis (second axis Ax2) of sphere bearing 2c, that is, so as to maintain coaxiality.

Traction sections 8 are two disk-like sections which form a substantially circular shape when viewed from the front side in the drawing, and are configured to have a base portion 8c, which is provided on the rear side in the drawing, and rotating portion 8d, which is provided on the front side in the drawing. Base portion 8c of traction section 8 is supported by being engaged with sphere bearing 2c on the spherical surface from the rear side in the drawing. That is, sphere bearing 2c supports traction section 8 (base portion 8c) with respect to the surface orthogonal to the axis (second axis Ax2) of straight portion 4 so as to be inclined in an arbitrary direction while restricting the movement of the entirety of traction section 8 in the frontward-rearward direction. In addition, rotating portion 8d of traction section 8 is configured so as to be rotatable relatively to base portion 8c.

Rotating portion 8d of traction section 8 is provided with, for example, a columnar guide piece 8a, which extends in a circumferential direction of traction section 8, at symmetric positions by interposing second axis Ax2. For example, a rectangular column-like guide section 4c is formed at the proximal end (rear end) of straight portion 4 so as to protrude rearward in the drawing, an elliptic guide hole 4b is provided in guide section 4c, and guide piece 8a of traction section 8 is engaged with guide hole 4b of straight portion 4 so as to be movable with respect to the guide hole. Through the engagement between guide piece 8a and guide hole 4b, traction section 8 is configured so as to be supported on the proximal side of straight portion 4 and relatively displaceable (inclinable) with respect to the axis (second axis Ax2) of straight portion 4. For this reason, rotating portion 8d is inclinable with respect to the surface orthogonal to the axis of straight portion 4, and rotates together with straight portion 4 in an inclined state.

As described above, straight portion 4 maintains coaxiality with sphere bearing 2c due to connection part housing 3a and traction section 8 is inclinable due to sphere bearing 2c. Therefore, according to this configuration, the inclination direction and the inclination angle of traction section 8 in connection part 3 change in a state while the relative positional relationship between grip part 2 (sphere bearing 2c) and straight portion 4 does not change on the front side and the rear side of connection part 3 (that is, while the coaxiality of the grip part and the straight portion is maintained). However, the direction in which traction section 8 is inclinable is limited due to the provision of the engagement structure formed by guide piece 8a and guide hole 4b. Here, as can be understood from the relationship between FIG. 2 and FIG. 3, only inclination of traction section 8 having third axis Ax3 (rotary axis of traction section 8), which is shown in FIG. 3, is allowed. The maximum value of angle θ at this time is substantially determined by the length of guide hole 4b in the frontward-rearward direction, in the engagement structure.

A part (upper part in the drawing) of the vicinity of the outer circumferential portion of traction section 8 is biased rearward in the drawing at all times by biasing section 8b which is formed of an elastic body such as a coil spring, in connection part 3. In addition, in traction section 8, the position (lower part in the drawing) on a side opposite to biasing section 8b by interposing the central portion (second axis Ax2) which is engaged with sphere bearing 2c is pulled rearward by first operation part 2a through link section 10.

In addition, in the outer circumferential portion of traction section 8, a starting end of first operation wire 20a is fixed to a part (upper part in the drawing) and a starting end of second operation wire 20b is fixed to the position (lower part in the drawing) on a side opposite to biasing section 8b by interposing the central portion (second axis Ax2) of traction section 8 (hereinafter, in some cases, these wires are collectively called operation wires 20). It is possible to favorably use, for example, a twisted thread such as a stainless wire as operation wires 20.

The starting end sides of operation wires 20 are pulled rearward by traction section 8. In a basic structure of operation wires 20, the starting ends of first operation wire 20a and second operation wire 20b are respectively fixed to areas which are separated from each other by 180° in a circumferential direction by interposing second axis Ax2, in the outer circumferential portion of traction section 8. Wire guide 9 is provided in front of traction section 8 in accordance with the fixed positions of the operation wires 20. Wire guide 9 is relatively indisplaceably fixed to, for example, the vicinity of guide section 4c at the proximal portion of straight portion 4, and is configured to have first fixed pulley 9aa which is provided on an outer circumferential side and second fixed pulley 9ab which is provided on an inner circumferential side.

The extending direction of operation wire 20 is first changed from the outer circumferential side to the inner circumferential side by first fixed pulley 9aa, and is next changed from the rear side to the front side in the drawing by second fixed pulley 9ab. Operation wire 20 of which the extending direction is changed to the front side in the drawing by second fixed pulley 9ab is guided to proximal end 5a of bending portion 5 through the inside of hollow portion 4a of cylindrical straight portion 4, and then, is guided to free end 5b side of bending portion 5 sequentially via through-holes which are provided inside joint pieces 30 of bending portion 5 and are not shown in the drawing. The terminal end of first operation wire 20a is fixed to first fixing point 5d which is provided on an upper side of bending portion 5 in the drawing, on an inner surface of bending portion 5 on the free end 5b side. Similarly, second operation wire 20b is fixed to second fixing point 5e which is provided on a lower side of bending portion 5 in the drawing.

When operating force is imparted rearward to a lower part of traction section 8 in the drawing by operating first operation part 2a in the initial state shown in FIG. 2, traction section 8 is inclined by angle θ with respect to the surface orthogonal to second axis Ax2 by having third axis Ax3 as an axis, in accordance with the operation amount of first operation part 2a as shown in FIG. 3. Second operation wire 20b is pulled rearward in accordance with the inclination of traction section 8, and second fixing point 5e is pulled on free end 5b of bending portion 5, which is then finally curved downward in the drawing. At this time, first operation wire 20a which is connected to first fixing point 5d is reeled out forward in accordance with the curving of bending portion 5.

The length (hereinafter, referred to as “traction amount”) of operation wire 20 which is pulled out by traction section 8 is determined by the inclination angle of traction section 8 which has third axis Ax3 as an axis, the position in which the starting end of operation wire 20 is fixed to traction section 8, and the distance to third axis Ax3 (more precisely, an intersection point of the surface to which the starting end of operation wire 20 is fixed, and second axis Ax2). Accordingly, the traction amount is increased by increasing the outer diameter of traction section 8, and therefore, it is possible to increase the curving angle of bending portion 5. Connection part housing 3a in which traction section 8 is accommodated is on the outside of the body, and therefore, the size of the outer diameter thereof is not particularly limited.

In the present embodiment, the state in which bending portion 5 is not curved as shown in FIG. 2 is set to an initial state. However, a state in which bending portion 5 is curved upward by adjusting a tension of biasing section 8b may be set to an initial state. By doing this, bending portion 5 enters the linear state shown in FIG. 2 from a state in which the bending portion is curved upward in the drawing through an operation of first operation part 2a. Furthermore, it is possible to displace the bending portion to a state in which the bending portion is curved downward in the drawing as shown in FIG. 3 by further adding the operation of the first operation part.

In addition, in the configuration shown in the drawing, the upper part of traction ection 8 is set to be biased rearward by biasing section 8b, in connection part housing 3a. However, the upper part of traction section 8 may have a vertical push-pull configuration by being combined with link section 10. By doing this, it is possible to curve bending portion 5 upward in the drawing, from the initial state shown in FIG. 2 by pulling first operation wire 20a based on the operation of first operation part 2a.

In addition, bending portion 5 may be configured so as to independently maintain the linear state (or the above-described state in which the bending portion is curved upward in the drawing) as an initial state by, for example, connecting adjacent joint pieces 30 to each other using an elastic section such as a spring, in bending portion 5. In this case, when operation wire 20 is not pulled, bending portion 5 returns to the initial state due to the elasticity which is possessed by the bending portion itself. Therefore, at least one operation wire 20 is sufficient while the curving direction of bending portion 5 is limited to one direction.

In the above-described basic configuration, a configuration example has been shown in which bending portion 5 can be curved in one direction by providing two operation wires 20 as first power transmission sections. However, the curving direction of bending portion 5 is not limited thereto. For example, a configuration may be employed in which three or four operation wires are provided and bending portion 5 is curved in three or four directions. For example, it is possible to have a configuration in which the bending portion can be curved within a range of ±90° of a curving angle in an X-axis direction and a Y-axis direction orthogonal to each other.

Next, a configuration relating to a tilting operation in hard part 6 will be described with reference to FIGS. 4 to 6B. Here, a configuration example is shown in which the direction of the visual field of imaging unit 6a is made to be displaceable by 90°.

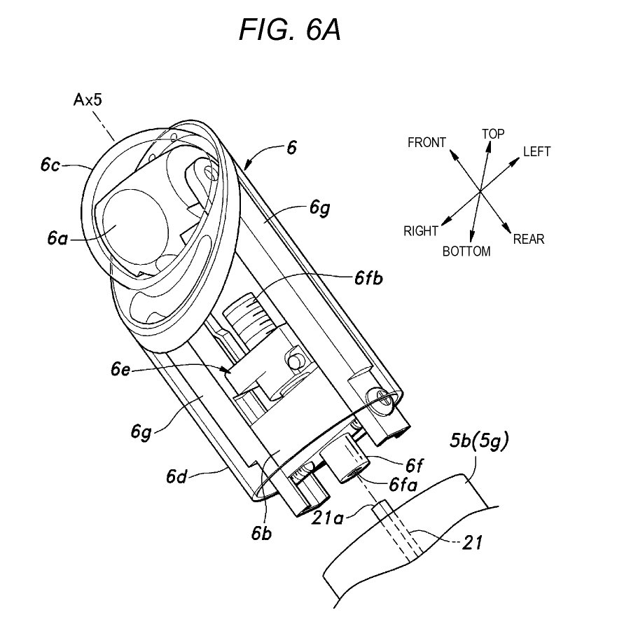

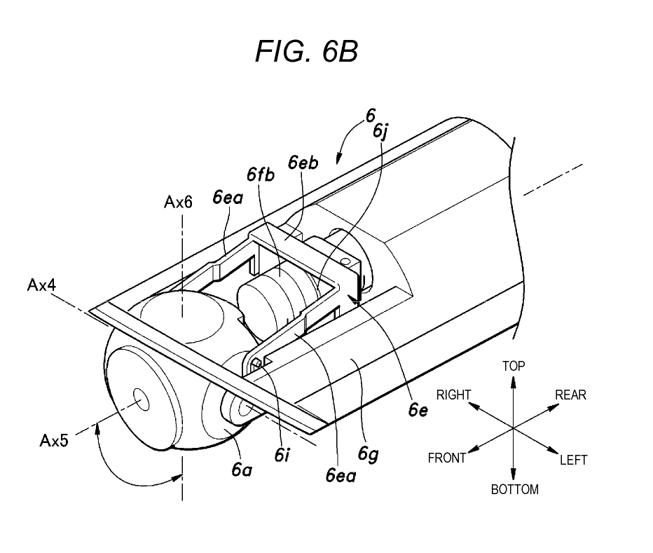

FIGS. 6A and 6B are transparent views showing a configuration of hard part 6 which is attached to bending portion 5 on a free end 5b side. Hard part 6 has camera support body 6b, camera outline 6d, imaging unit 6a, functional section-displacement portion 6e for displacing imaging unit 6a, and shaft coupling-engaged portion 6f which transmits driving force from the outside to functional section-displacement portion 6e.

Camera outline 6d is a section which is formed in a substantially cylindrical shape, and is formed of, for example, stainless steel. The length of camera outline 6d in a frontward-rearward direction (longitudinal direction) is formed to be long on an upper side in the direction shown in the drawing and short on a lower side in the direction shown in the drawing. A cut surface which is obliquely cut with respect to the frontward-rearward direction is formed at a tip end of camera outline 6d. Hemispherical transparent dome 6c is provided on the cut surface of camera outline 6d and imaging unit 6a is provided in dome 6c.

Imaging unit 6a has an image pickup device (not shown) which is formed of a compact charge coupled device (CCD), a complementary metal-oxide semiconductor (CMOS), or the like; and an optical lens (not shown) that forms an image of object light, which is incident through dome 6c, on the image pickup device. Imaging unit 6a is pivotally supported by a support arm 6g which extends in the frontward-rearward direction, so as to be rotatable on both sides in a right-left direction of camera support body 6b shown in the drawing. Imaging unit 6a with such a shape can be easily realized by, for example, utilizing a camera module which is used in smart phones or tablet terminals.

Functional section-displacement portion 6e is a driving section which is formed substantially in a U-shape, and has driving arms 6ea which extend to both sides of imaging unit 6a along the inside of support arm 6g; and arm support body 6eb which supports driving arms 6ea from the rear side. Imaging unit 6a is provided with an engagement portion 6i which protrudes at a position which is offset in a radial direction from fourth axis Ax4 which becomes a rotary axis of imaging unit 6a, and driving arms 6ea are engaged on both sides of imaging unit 6a in engagement portion 6i. Screw hole 6j which penetrates in the frontward-rearward direction shown in the drawing is provided substantially at the center of arm support body 6eb in a front view, and shaft coupling-engaged portion 6f is inserted and screwed into screw hole 6j.

Shaft coupling-engaged portion 6f is exposed from a rear end of camera support body 6b by penetrating camera support body 6b, and square hole 6fa which is recessed forward as shown in the drawing is provided at the center at a rear end of shaft coupling-engaged portion 6f. Shaft coupling-engaged portion 6f is engaged with and connected to shaft coupling engaging portion 21a at a tip end of torque tube 21 which protrudes on the free end 5b side of bending portion 5 in square hole 6fa so as to be rotatable together with shaft coupling engaging portion 21a.

The front part of shaft coupling-engaged portion 6f constitutes lead screw 6fb inside of hard part 6. Lead screw 6fb is screwed into screw hole 6j which is formed in arm support body 6eb, and lead screw 6fb (shaft coupling-engaged portion 6f) is rotated around fifth axis Ax5 (return angle of shaft coupling-engaged portion 6f). Accordingly, driving arms 6ea which are provided in arm support body 6eb move in the frontward-rearward direction, which is shown in the drawing, along support arm 6g. In this manner, functional section-displacement portion 6e converts a rotary motion which is received by shaft coupling-engaged portion 6f into a linear motion. Imaging unit 6a is driven in the frontward-rearward direction in engagement portion 6i in accordance with the movement in the frontward-rearward direction of driving arm 6ea. Accordingly, imaging unit 6a rotates around an axis which is pivotally supported by support arm 6g, that is, fourth axis Ax4.

At this time, shaft coupling-engaged portion 6f rotates together with shaft coupling engaging portion 21a of torque tube 21 by rotating shaft coupling engaging portion 21a around fifth axis Ax5 as an axis, and therefore, lead screw 6fb rotates. Imaging unit 6a rotates around fourth axis Ax4 and is displaced (rotated) in an inclined direction with respect to fifth axis Ax5. Accordingly, the imaging direction due to imaging unit 6a changes at least from the forward (fifth axis Ax5) direction to the downward (sixth axis Ax6 orthogonal to fifth axis Ax5) direction in the directions shown in the drawing, and therefore, the movement of the visual field in the vertical direction, that is, the tilting operation is realized. The amount of the visual field moved in the frontward-rearward direction with respect to the rotation amount is set by appropriately setting groove pitch or the like of the above-described lead screw 6fb. Therefore, it is possible to precisely adjust the rotation angle of imaging unit 6a which has been pivotally supported.

In the above-described embodiment, an example has been shown in which imaging unit 6a itself that is provided with an image pickup device and an optical lens as mechanisms for the tilting operation that moves the visual field is configured so as to rotate around the pivotally supported axis. However, the present invention is not limited thereto. For example, a configuration may be set in which the image pickup device is fixed to hard part 6 and the optical path of subject light is changed by rotating an optical section such as a mirror section which is provided between the image pickup device and the optical lens.

Next, a configuration in which driving force to be supplied for a tilting operation is transmitted to hard part 6 will be described. As shown in FIGS. 4 and 5, torque tube 21 extends between grip part 2 and hard part 6. Torque tube 21 is mechanically connected to second operation part 2b (refer to FIG. 1) through a gear train or the like which is not shown in the drawing, on the grip part 2 side. Torque tube 21 rotates around second axis Ax2 as an axis by operating second operation part 2b. At this time, the gear train which is provided in grip part 2 converts the rotating operation around first axis Ax1 (refer to FIG. 1) of second operation part 2b to a plurality of rotating motions. As described above, lead screw 6fb is rotated by the rotation of torque tube 21, the rotating force is converted to a linear motion by functional section-displacement portion 6e, and imaging unit 6a is rotated. Torque tube 21 may be accommodated in a flexible pipe which extends between grip part 2 and hard part 6.

Bearing opening 2d and traction section opening 8e are respectively formed at a tip end of sphere bearing 2c, which is provided in grip part 2, and at a central portion in a radial direction of fraction section 8 which is provided in connection part 3. Torque tube 21 extends forward in the drawing along second axis Ax2 through the inside of hollow portion 4a of straight portion 4 via bearing opening 2d and traction section opening 8e. For this reason, rotating force which is transmitted by torque tube 21 is set so as not to interfere with each other between traction sections 8.

Even in bending portion 5, torque tube 21 extends along the axis of bending portion 5 through hollow portion 5c of bending portion 5. In order to position torque tube 21 at the center in the radial direction of hollow portion 5c even in a state where bending portion 5 is curved as shown in FIG. 5, an intermediate support section which is not shown in the drawing may be provided in all or part of joint pieces 30 and torque tube 21 is made to pass through a through-hole of the intermediate support section.

FIG. 4 shows a state in which bending portion 5 is in a linear shape in the second axis Ax2 direction (that is, hard part 6 faces forward in the drawing) and the visual field of imaging unit 6a which is provided in hard part 6 faces forward in the drawing. When an operator or the like operates second operation part 2b (refer to FIG. 1) of grip part 2 in the state shown in FIG. 4, torque tube 21 rotates in accordance with the operation amount. Accordingly, imaging unit 6a which is pivotally supported by hard part 6 rotates. Accordingly, the visual field moves from the forward direction (direction of fifth axis Ax5) in the drawing to the downward direction (direction of sixth axis Ax6) in the drawing. In the present embodiment, the rotating angle of imaging unit 6a is set to 90° at maximum, and therefore, an operator or the like can arbitrarily adjust the rotating angle of imaging unit 6a, that is, the tilt angle, between fifth axis Ax5 and sixth axis Ax6.

When an operator or the like operates first operation part 2a of grip part 2 from the state shown in FIG. 4, traction section 8 is inclined in accordance with the operation amount. Accordingly, operation wire 20 (here, second operation wire 20b) is pulled, and bending portion 5 is curved from the forward direction (direction of second axis Ax2) in the drawing to the downward direction (direction of seventh axis Ax1) in the drawing as shown in FIG. 5. In the present embodiment, the curving angle of bending portion 5 is set to 90° at maximum, and therefore, an operator or the like can arbitrarily adjust the curving angle of bending portion 5, between second axis Ax2 and seventh axis Ax1.

Furthermore, even in the state shown in FIG. 5, an operator or the like can arbitrarily adjust the tilt angle, between fifth axis Ax5 and sixth axis Ax6. Here, direction D2, in which bending portion 5 is curved, and direction D3, in which pivotally supported imaging unit 6a is rotated (tilted) are set to be substantially the same as each other. Therefore, it is possible to move the visual field within a range of 180° from the forward direction (second axis Ax2) in the drawing to the rearward direction (sixth axis Ax6) in the drawing through the curving operation of bending portion 5 and the tilting operation of imaging unit 6a. That is, according to the configuration of the present embodiment, it is unnecessary to increase the curving angle of bending portion 5, and therefore, it is possible to move the visual field within a wide range even if bending portion 5 is formed to be short. In addition, the curving angle of bending portion 5 is sufficiently small, and therefore, it is possible to use endoscope 1 even in a narrow space. Furthermore, wearing or the like of operation wire 20 is reduced by making the curving angle of bending portion 5 small, and therefore, it is possible to maintain the reliability of the operation wire over a long period of time.

In the present embodiment, a first power transmission section (operation wire 20) that transmits operating force, which is generated on the proximal end 5a side of bending portion 5, to the free end 5b side as a traction force in order to curve a bending portion 5; and a second power transmission section (torque tube 21) that transmits operating force, which is generated on the proximal end 5a side, to the free end 5b side as rotating force, in order to displace (rotate) imaging unit 6a which is pivotally supported by hard part 6 as a functional section extend to hollow portion 5c of bending portion 5 from proximal end 5a of bending portion 5 to free end 5b.

As described above, operation wire 20 is disposed along the inner surface of bending portion 5 and torque tube 21 is disposed substantially at the center of bending portion 5 in a radial direction. Accordingly, the path length of torque tube 21 which is disposed at the center of bending portion 5 in the radial direction does not change even if bending portion 5 is curved by operating operation wire 20. Therefore, it is possible to stably transmit driving force (rotating force) used for a tilting operation of imaging unit 6a through shaft coupling engaging portion 21a (refer to FIG. 6A), which is provided at the tip end of torque tube 21, and shaft coupling-engaged portion 6f (refer to FIG. 6A), which is provided at the rear end of hard part 6, being stably engaged at all times. That is, according to the present embodiment, the paths of operation wire 20 and torque tube 21 in bending portion 5 are completely separated from each other and the driving force to be supplied for the movement of the visual field is transmitted by being separated into different types of transmission systems such as traction force and rotating force. Therefore, it is possible to secure independence of a curving operation and a tilting operation by preventing mutual interference between a first power transmission section and a second power transmission section.

In the above-described description, torque tube 21 has been exemplified as a second power transmission section which transmits driving force of a tilting operation. However, a flexible rod-like section may be configured as the second power transmission section. In addition, the rod-like section may be divided in a longitudinal direction so as to be made into a plurality of divided pieces, each of which may then be combined by a joint by employing the same structure as that of the above-described bending portion 5.

In addition, a configuration of an operation part of operating a tilting operation using second operation part 2b which is provided in grip part 2 has been exemplified. However, an operation ring may be provided between rotating operation part 7, which rotates the entire insertion section 11, and a proximal portion of straight portion 4, and torque tube 21 may be rotated by a rotating operation of the operation ring to perform a tilting operation of imaging unit 6a.

Next, a configuration relating to the rotating operation of hard part 6 will be described with reference to FIGS. 3 and 5.

Rotating operation part 7 which rotates straight portion 4 around second axis Ax2 is fixed to an outer circumference of straight portion 4. However, the rotation of rotating operation part 7 and straight portion 4 is limited. That is, a stopper which is not shown in the drawing is provided in grip part 2, and only one turn of rotation (alternately, a clockwise half turn or a counterclockwise half turn) of rotating operation part 7 at maximum is allowed by the restriction of the stopper. A transmission cable or the like which extends along an axial direction of bending portion 5 and is not shown in the drawing is prevented from being excessively twisted, by restricting the rotation of straight portion 4 in this manner.

In addition, as described above, base portion 8c of traction section 8 is fixed by sphere bearing 2c so as to be able to be inclined in an arbitrary direction to the surface orthogonal to second axis Ax2 and to be unable to rotate around second axis Ax2 as an axis. In contrast, rotating portion 8d of traction section 8 is configured so as to be rotatable in a direction which is inclined by angle θ as an axis from second axis Ax2. In addition, guide hole 4b which forms a long hole in the frontward-rearward direction in the drawing is provided in the vicinity of the rear end of straight portion 4 and guide piece 8a of traction section 8 is guided such that traction section 8 can be relatively inclined with respect to straight portion 4.

When rotating operation part 7 which is fixed to the outer circumference of straight portion 4 is rotated around second axis Ax2 as an axis in a state where bending portion 5 is curved as shown in FIGS. 3 and 5, straight portion 4 rotates around second axis Ax2 as an axis along with the rotation of rotating operation part 7. The rotation is transmitted to rotating portion 8d of traction section 8 through guide hole 4b which is provided in straight portion 4 and guide piece 8a which is provided in fraction section 8. Rotating portion 8d is freely rotatable with respect to base portion 8c of traction section 8, and therefore, rotates around an axis which is inclined by angle θ with respect to second axis Ax2 as a rotary axis.

When rotating portion 8d rotates, operation wire 20, of which a starting end is fixed to rotating portion 8d, and wire guide 9 which is fixed to straight portion 4 also rotate at the same time in accordance with the rotation. When traction section 8 rotates, the inclination direction and the inclination angle of base portion 8c when viewed from a predetermined direction orthogonal to the axis direction (second axis Ax2) of straight portion 4 is maintained. Accordingly, even when rotating portion 8d which is supported by base portion 8c so as to be rotatable rotates, the inclination direction and the inclination angle of rotating portion 8d are maintained. That is, in regard to the inclination direction and the inclination angle of traction section 8, the state shown in FIG. 3 is maintained even if rotating operation part 7 is rotated, and therefore, the traction amounts of a plurality of operation wires 20 which is pulled by traction section 8 together with the rotation of rotating portion 8d change, and the bending portion 5 maintains the state of being curved downward in the drawing. That is, when rotating operation part 7 is rotated, free end 5b of bending portion 5 is rotated around fifth axis Ax5 as an axis in direction D1.

Hard part 6 which is attached to the free end 5b side also rotates due to the rotation of free end 5b of bending portion 5. When the initial imaging direction due to imaging unit 6a is set to be sixth axis Ax6 direction (rearward direction in the drawing) shown in FIG. 5, the visual field moves in direction D1 (circumferential direction) by having fifth axis Ax5 as an axis. That is, in the present embodiment, when bending portion 5 rotates, imaging unit 6a as a functional section, a plurality of operation wires 20, and traction section 8 rotate together with bending portion 5, and traction section 8 changes the traction amount with respect to the plurality of operation wires 20 to maintain the curving direction and the curving angle of bending portion 5. This is an operation corresponding to “panning” in camera work, and hereinafter, the operation of moving the visual field in the circumferential direction is called a “panning operation” or is simply called “panning”.

In addition, when the initial imaging direction is fifth axis Ax5, an imaged image rotates around an optical axis due to rotation of free end 5b of bending portion 5. This is an operation corresponding to “rolling” in camera work, and hereinafter, the operation of rotating an image around an optical axis is called a “rolling operation” or is simply called “rolling”. The “panning operation” and the “rolling operation” are collectively called a “panning operation or the like”.

In the present embodiment, the panning operation and the rolling operation which is not affected by the curving direction and the curving angle of bending portion 5 and in which straight portion 4 is rotated are performed as simple and intuitive operations. In addition, the outer diameter of the above-described rotating operation part 7 is configured to be larger than that of straight portion 4, and the operability is improved by performing these operations with smaller force.

As described above, in endoscope 1 of the present embodiment, bending portion 5 can be curved in an arbitrary direction in the lumen in a living body or the like, and imaging unit 6a which is provided on the free end 5b side of bending portion 5 performs tilting and panning operations or the like. Accordingly, the degree of freedom of the visual field operation performed by an operator is greatly improved, and it is possible to apply endoscope 1 of the present embodiment to various operative procedures. Moreover, it is possible to perform all of the operations such as curving, tilting, panning, and rolling, and therefore, it is possible to perform safer surgery or the like.

In addition, surgical instruments such as forceps or a laser scalpel in addition to endoscope 1 are inserted into the lumen in a living body during surgery. In some cases, the direction in which a laser scalpel is to be moved and the direction of an image which is imaged by endoscope 1 do not coincide with each other depending on the positional relationship between endoscope 1 and other instruments (for example, in a case where a tip end of hard part 6 of endoscope 1 and a tip end of the laser scalpel are in a positional relationship in which they face each other). According to the present embodiment, when the imaging direction using imaging unit 6a is set to fifth axis Ax5 direction as in FIG. 5, it is possible to rotate (vertically reverse) the image around the optical axis by rolling hard part 6. Accordingly, it is possible to make the top and bottom (right and left) coincide with each other at all times in the operation direction of other instruments and the image, and therefore, it is possible to secure safety during surgery or the like. The vertical reversal (180° top-bottom reversal) can be satisfied with simple image processing. However, when the rotation angle of the image is arbitrarily set, image pixels are generated through interpolation in the image processing. Therefore, the resolution deteriorates, particularly when the number of image pixels of an image pickup device is low. From this point, in endoscope 1 of the present embodiment, since imaging unit 6a itself is rolled, the resolution does not deteriorate.

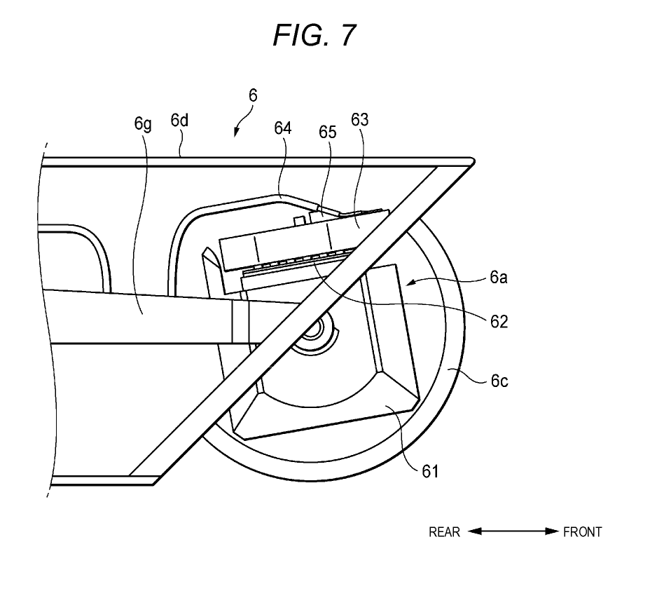

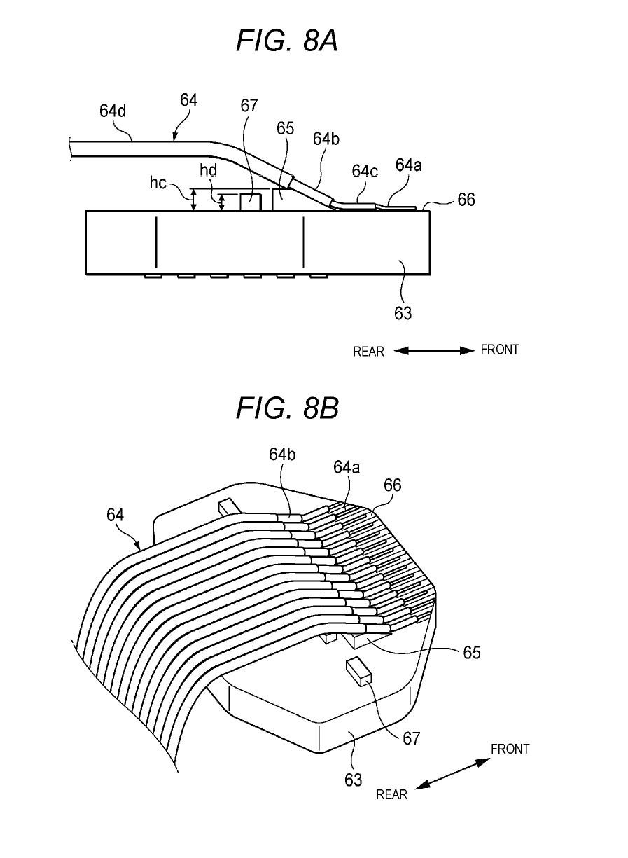

Next, the detailed configuration of imaging unit 6a in hard part 6 will be described with reference to FIGS. 7 to 8B. FIG. 7 is a transparent view showing an outline of an arrangement configuration of imaging unit 6a in hard part 6. Some parts are hidden for description. FIGS. 8A and 8B are views showing a first configuration example of a connection portion between board 63 and cable 64 in imaging unit 6a. FIG. 8A is a side view and FIG. 8B is a perspective view.

Imaging unit 6a is formed so as to be rotatably attached to support arm 6g in the inside of hard part 6 at the tip end portion of the insertion section; be able to rotate around an axis orthogonal to the direction of a longitudinal axis of the insertion section; and be able to displace the visual field direction. Imaging unit 6a has optical lens unit 61 which forms an object image; image pickup device 62 using a CCD, a CMOS, or the like; and board 63 for assembling image pickup device 62. Image pickup device 62 is installed on one surface of board 63. One end (distal portion on the imaging unit) of cable 64 for performing input and output of a signal with respect to board 63 is assembled on and fixed to the other surface of board 63 which is electrically connected to a circuit of board 63 and image pickup device 62.

Cable 64 is configured such that, for example, a plurality of coaxial cables are disposed in a belt shape, and has core line (inside conductor) 64a at a central portion; shield part (outside conductor) 64b on the outside; insulator 64c that insulates core line 64a and shield part 64b; and coating 64d in an outer circumferential portion. Cable 64 may be either a cable in which a single coaxial cable is separated in pieces, a cable in which an end portion of a single coaxial cable is fixed and arranged together, or a flat cable-like cable in which coaxial cables are connected to each other. As an example of cable 64, a thin-wire coaxial cable at AWG 46 of which the diameter of a core line is about 0.041 mm is used.

When imaging unit 6a of the present embodiment is rotated inside hard part 6, cable 64 can be flexibly deformed in accordance with the rotation of imaging unit 6a and board 63 and optical lens unit 61 can be smoothly displaced. On board 63 it is possible to secure the space for rotating imaging unit 6a and to minimize the size of hard part 6, by reducing the size of board 63 as much as possible in addition to devising the shape thereof by obliquely cutting the corners or the like. Here, improvement in assembling efficiency by increasing an assembling possible area of an assembly section on board 63 contributes to the miniaturization of board 63.

In a cable-mounting surface of board 63, ground bar 65 for grounding shield part 64b of cable 64 is provided on a board-mounting portion of cable 64. Ground bar 65 is formed of, for example, a conductor such as a metal section; is assembled in a state of protruding from the surface of board 63; and is electrically connected to a ground pattern of board 63. As a material of ground bar 65, for example, phosphor bronze is used. Ground bar 65 may be assembled on board 63 through soldering or the like and may be configured to be installed in a state where a part of the ground bar is embedded in the board. In addition, a pad 66 which becomes a cable connection terminal is provided on the cable-mounting surface of board 63.

In cable 64, shield part 64b is electrically connected and attached to ground bar 65 and core line 64a is electrically connected and attached to pad 66, through soldering or the like. That is, in the distal portion of cable 64 on an imaging unit side, shield part 64b is connected to a ground of board 63 through ground bar 65 and core line 64a is directly assembled on pad 66 of board 63. For example, electronic component 67 such as a capacitor, a resistor, or a chip component is assembled on the cable-mounting surface of board 63 as an assembly section.

Ground bar 65 is a thin and long rod-like conductive section, and has an inclination surface which is inclined such that the height of the ground bar is lowered toward the pad 66 side (distal side of cable 64). Moreover, the cross-sectional shape of a cut surface which is orthogonal to a longitudinal direction is formed in a substantially triangular shape or a substantially trapezoidal shape. Shield part 64b of cable 64 is brought into contact and conducted with ground bar 65 along the inclination surface of the ground bar and is connected to the inclination surface through soldering or the like. At this time, shield part 64b of cable 64 is connected to the ground pattern of board 63 through ground bar 65 without being directly connected to board 63, and therefore, the ground of cable 64 is secured. Ground bar 65 supports cable 64 on board 63 such that the height of the cable in the vicinity of the board-mounting portion of cable 64 becomes greater than or equal to a predetermined value.

Height hc of ground bar 65 from the surface of board 63 to cable 64 on the rear end side (proximal side of the insertion section and rear side in the drawing) can secure a height greater than or equal to a predetermined value by assembling cable 64 on board 63 by providing ground bar 65. In the present embodiment, height hc of cable 64 from the surface of board 63 of cable 64 at a rear end portion of the ground bar 65 in the drawing, that is, at a side end portion on a side opposite to the distal portion of cable 64 and the vicinity of the side end portion is set to be higher than height hd of electronic component 67 which is assembled on board 63 and is positioned nearest to the cable.

In this manner, it is possible to raise the height of cable 64 using ground bar 65 and to increase routing of cable 64 assembled on board 63 more than that of electronic component 67. For example, when electronic component 67 is a chip component with a 0603 size, height hd of the component is about 0.25 mm. Therefore, height hc (raising height of cable 64) of cable 64 at the rear end portion of ground bar 65 in the drawing may be set to be greater than or equal to 0.25 mm.

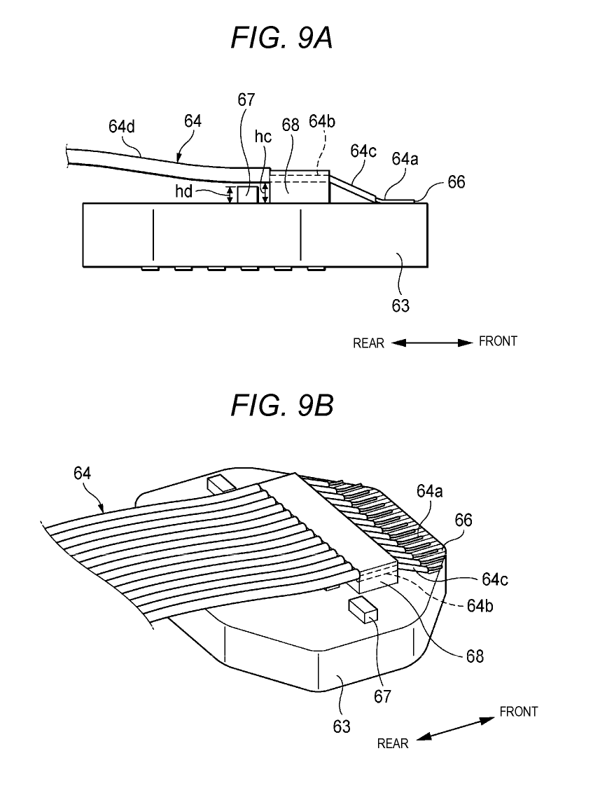

FIGS. 9A and 9B are views showing a second configuration example of a connection portion between board 63 and cable 64 in an imaging unit 6a. FIG. 9A is a side view and FIG. 9B is a perspective view.

The second configuration example shown in FIGS. 9A and 9B is an example in which the configuration of ground bar 68 is changed. Ground bar 68 is a thin and long rod-like conductive section, and does not have an inclination surface. Moreover, the cross-sectional shape of a cut surface which is orthogonal to a longitudinal direction is formed in a substantially rectangular shape. Ground bar 68 may be configured so as to connect and fix shield part 64b of cable 64 to the upper surface thereof as a flat prismatic section, or may be configured so as to connect and fix shield part 64b of cable 64 to an insertion hole by providing the insertion hole in a frontward-rearward direction in the drawing and inserting an end portion of cable 64.

In the second configuration example, it is also possible to make height hc of ground bar 68, of which the cross-sectional shape is a substantially rectangular shape, from the surface of board 63 of cable 64 in a rear end portion in the drawing higher than height hd of electronic component 67 nearest to the cable, and therefore, it is possible to secure the height greater than or equal to a predetermined value.

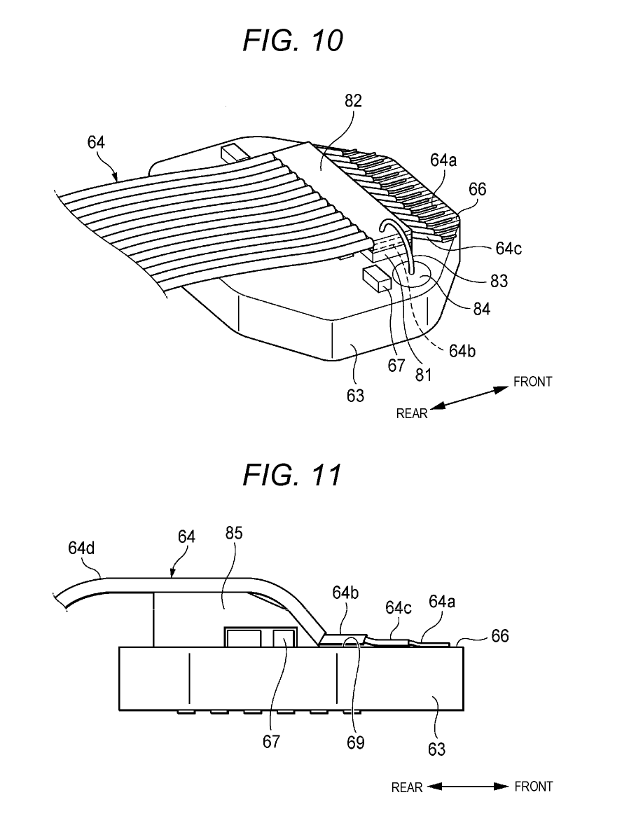

FIG. 10 is a perspective view showing a third configuration example of a connection portion between board 63 and cable 64 in imaging unit 6a.

The third configuration example shown in FIG. 10 is an example in which a raising section 81 is provided using an insulation section instead of ground bar 68. Raising section 81 is a thin and long rod-like section and is constituted of an insulation section such as a resin section. The vicinity of a board-mounting portion of cable 64 is raised by raising section 81, and therefore, the height of cable 64 on board 63 is made to be higher than that of electronic component 67 which is positioned nearest to cable 64. Conductive section 82 is provided on the upper portion of raising section 81, and shield part 64b of cable 64 is connected thereto. Conductive section 82 is connected to land portion 84 on board 63 through ground wire 83 and is electrically connected to the ground pattern of board 63.

In the third configuration example, it is possible to make the height of cable 64 from the surface of board 63 in a rear end portion of raising section 81 in the drawing higher than that of electronic component 67 nearest to the cable, and therefore, it is possible to secure the height greater than or equal to a predetermined value.

FIG. 11 is a side view showing a fourth configuration example of a connection portion between board 63 and cable 64 in imaging unit 6a.

The fourth configuration example shown in FIG. 11 is an example in which cable 64 is directly assembled on board 63 and raising section 85 is provided on a proximal side (rear side in the drawing) of a cable-mounting portion. Shield part 64b and core line 64a of cable 64 are respectively attached to land portion 69 and pad 66 on board 63 by being electrically connected thereto through soldering or the like. Land portion 69 is connected to a ground pattern of board 63. Raising section 85 is attached to the further proximal side (rear side in the drawing) of the insertion section than land portion 69. Raising section 85 is constituted of an insulation section such as a resin section; has an inclination surface similar to ground bar 65; and is made to be a guide section for securing the height of cable 64 on the board. The vicinity of a board-mounting portion of cable 64 is raised by raising section 85, and therefore, the height of cable 64 on board 63 is made to be higher than that of electronic component 67 which is positioned nearest to cable 64.

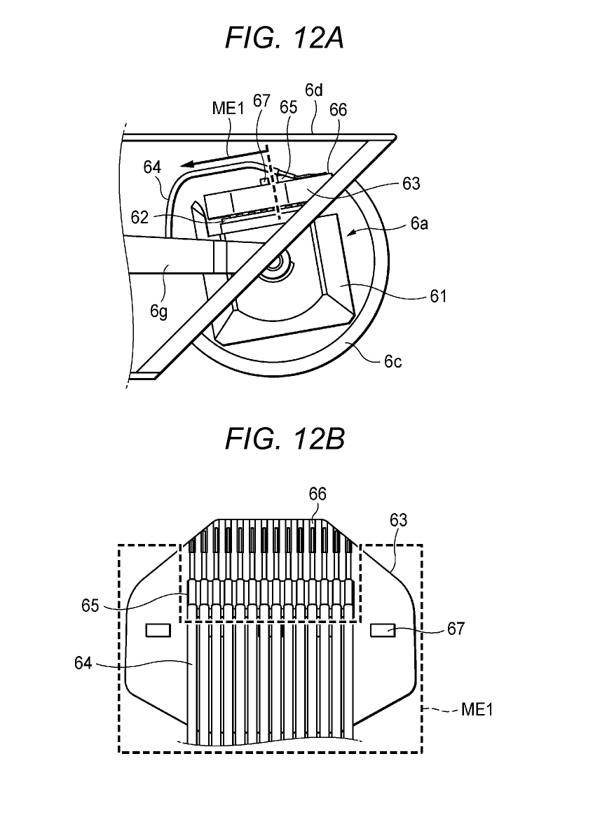

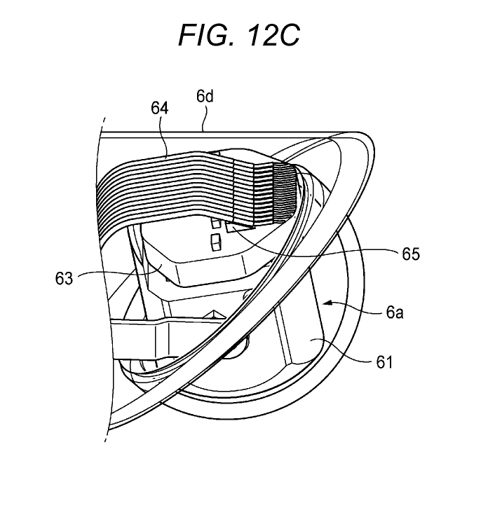

In the fourth configuration example, it is also possible to make the height of cable 64 from the surface of board 63 in a rear end portion of raising section 85 in the drawing higher than that of electronic component 67 nearest to the cable, and therefore, it is possible to secure the height greater than or equal to a predetermined value. FIGS. 12A to 12C are views for illustrating an assembling possible area of a component in board 63 of imaging unit 6a of the present embodiment. FIG. 12A is a transparent side view, FIG. 12B is a plan view, and FIG. 12C is a transparent perspective view.

In the present embodiment, it is possible to secure the height of cable 64 using ground bar 65 to be greater than or equal to a predetermined value. Therefore, it is possible to assemble electronic component 67 in the immediate vicinity of ground bar 65. Accordingly, as shown in FIGS. 12A and 12B, it is possible to minimize the dead space on board 63 and to increase assembling possible area ME1. In this case, it is possible to set an area excluding an arrangement area of pad 66 and ground bar 65 on board 63 to assembling possible area ME1 and to maximize assembling possible area ME1.

According to the present embodiment, it is possible to achieve miniaturization of board 63 by reducing the dead space on board 63 and to realize further reduction in the diameter of a tip end portion of an insertion section of an endoscope even in a structure in which imaging unit 6a of the tip end portion of the insertion section rotates.

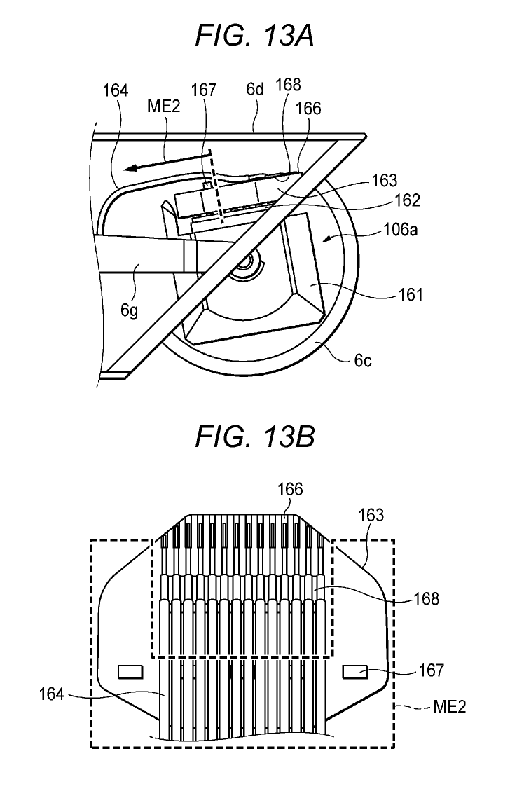

FIGS. 13A and 13B are views for illustrating an assembling possible area of a component in board 163 of an imaging unit of a comparative example. FIG. 13A is a transparent side view and FIG. 13B is a plan view.

Imaging unit 106a of the comparative example shown in FIGS. 13A and 13B has optical lens unit 161, image pickup device 162, and board 163. A ground bar is not provided on board 163 which is installed with image pickup device 162, and a core line and a shield part of cable 164 are respectively assembled on pad 166 for a signal line and pad 168 for a ground. In the comparative example, cable 164 is directly assembled on board 163 and the height of cable 164 is not secured. Therefore, as shown in FIGS. 13A and 13B, a dead space is generated in the vicinity of the portion assembled with cable 164 on board 163. In this case, it is impossible to assemble electronic component 167 in the vicinity of the portion assembled with cable 164, and therefore, assembling possible area ME2 on board 163 becomes small.

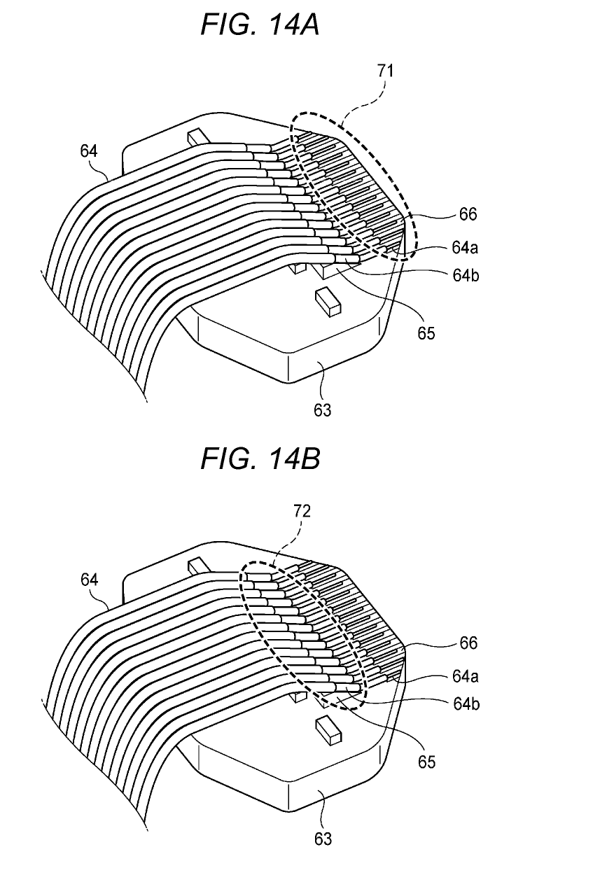

Next, a method of assembling cable 64 on a board in the present embodiment will be described. FIGS. 14A and 14B are views for illustrating an assembling procedure of cable 64 on board 63. FIG. 14A is a perspective view showing a first example of an assembling procedure. FIG. 14B is a perspective view showing a second example of an assembling procedure.

In the present embodiment, cable 64 and electronic component 67 are assembled on board 63 using a plurality of types of conductive connection materials such as solder or silver paste which have different melting points. When it is difficult to assemble assembly sections all at once through reflow, assembling is performed by being divided into a plurality of steps using conductive connection materials having different melting points. Accordingly, it is possible to suppress remelting of conductive connection materials during assembling in subsequent steps and to suppress positional deviation of assembly sections. Conductive connection materials including two types of conductive connection materials which have different melting points are applied on board 63 before assembling, and then, the assembling in a plurality of steps is performed by soldering through reflow, soldering through a manual operation, or the like.

In a first example of an assembling procedure shown in FIG. 14A, first, in a first step, assembly sections such as electronic component 67 are assembled using a reflow furnace and shield part 64b of cable 64 is assembled on ground bar 68. At this time, assembling is performed using, for example, tin-silver-copper based (Sn—Ag—Cu, melting point of 219° C.) solder as a first conductive connection material. Next, in a second step, core line 64a of cable 64 is assembled on pad 66 in a manual assembling area 71 of board 63 through a manual operation of an assembly worker. At this time, assembling is performed using, for example, tin-bismuth based (Sn—Bi, melting point of 136° C.) solder which has a lower melting point than that of the first conductive connection material, as a second conductive connection material. In the second step, core line 64a and pad 66 are connected to each other only through Sn—Bi in manual assembling area 71 being melted without Sn—Ag—Cu being remelted. In this manner, it is possible to appropriately assemble cable 64 and electronic component 67 through reflow and manual assembling using two types of conductive connection materials which have different melting points.

In a second example of an assembling procedure shown in FIG. 14B, first, in a first step, assembly sections such as electronic component 67 are assembled using a reflow furnace and core line 64a of cable 64 is assembled on pad 66. At this time, assembling is performed using, for example, tin-silver-copper based (Sn—Ag—Cu, melting point of 219° C.) solder as a first conductive connection material. Next, in a second step, shield part 64b of cable 64 is assembled on ground bar 68 in a manual assembling area 72 of board 63 through a manual operation of an assembly worker. At this time, assembling is performed using, for example, silver paste (Ag, melting point of 150° C.) which has a lower melting point than that of the first conductive connection material, as a second conductive connection material. In the second step, shield part 64b and ground bar 68 are connected to each other only through silver paste in manual assembling area 72 being melted without Sn—Ag—Cu being remelted. It is also possible to appropriately assemble cable 64 and electronic component 67 through reflow and manual assembling in the second example.

It is possible to perform assembling using three types of conductive connection materials which have different melting points by combining the above-described first example and second example, as a third example of an assembling procedure. In this case, for example, three types of conductive connection materials are used which satisfy T1>T2>T3, in which the melting point of the first conductive connection material is set to T1, the melting point of second conductive connection material is set to T2, and the melting point of third conductive connection material is set to T3. First, in a first step, assembly sections such as electronic component 67 are assembled by the first conductive connection material using a reflow furnace. Next, in a second step, shield part 64b of cable 64 is assembled on ground bar 68 by the second conductive connection material through reflow or a manual operation. Furthermore, in a third step, core line 64a of cable 64 is assembled on pad 66 by the third conductive connection material through a manual operation. In the third example, it is also possible to appropriately assemble cable 64 and electronic component 67 through reflow and a manual operation.

Mounting of various assembly sections such as the electronic component, the ground bar, and the cable can be performed by being divided into a plurality of steps such as reflow and a manual operation, using a plurality of types of conductive connection materials such as solder which have different melting points as in the present embodiment. In this case, it is possible to appropriately perform direct assembling on the substrate without causing a failure such as positional deviation due to remelting of conductive connection materials, using the conductive connection materials having different melting points.

In the configuration in which the inclination surface is provided on ground bar 65 and shield part 64b of cable 64 is connected to the inclination surface, when performing assembling of cable 64 through a manual operation, the assembling operation is easily performed by bringing core line 64a of cable 64 into contact with pad 66 and pressing shield part 64b of cable 64 on the inclination surface of ground bar 65. For this reason, the height of cable 64 is secured and the operability is improved.

Hereinabove, various embodiments have been described while referring to the drawings. However, needless to say, the present invention is not limited to the examples. It is obvious that various modification examples or revised examples can be conceived by those skilled in the art within the category disclosed in the claims, and it is naturally understood that the modification examples or the revised examples also belong to the technical scope of the present invention. In addition, the components in the above-described embodiment may be arbitrarily combined within a range not departing from the gist of the invention.

What is claimed is:

1. An endoscope comprising:
an imaging unit at a tip end portion of an insertion section, wherein the imaging unit has
a board on which the image pickup device is installed,
a cable through which a signal is input to and output from the board, and
a ground bar which is provided so as to protrude from the surface of the board and is connected to a ground of the board, and
wherein in a distal portion of the cable on the imaging unit side, a shield part is connected to the ground of the board through the ground bar and a core line is directly assembled on the board.

2. The endoscope of claim 1, wherein the height of the cable on the ground bar is set to be higher than that of an assembly section which is positioned nearest to the cable on the board.

3. The endoscope of claim 2, wherein the height of the cable on the board at a side end portion on a side opposite to the distal portion of the cable in the ground bar and in the vicinity of the side end portion is set to be higher than that of the assembly section which is positioned nearest to the cable.

4. The endoscope of claim 2, wherein the cross-sectional shape of a cut surface of the ground bar which is orthogonal to a longitudinal direction is formed in a substantially triangular shape or a substantially trapezoidal shape, and the ground bar has an inclination surface of which the height is lowered toward a distal side of the cable.

5. The endoscope of claim 2, wherein the cross-sectional shape of a cut surface of the ground bar which is orthogonal to a longitudinal direction is formed in a substantially rectangular shape.

6. The endoscope of claim 1, wherein an assembly section and the cable to be assembled on the board are assembled on the board using a plurality of types of conductive connection materials which have different melting points.

7. The endoscope of claim 1, wherein the imaging unit rotates around an axis orthogonal to the direction of a longitudinal axis of the insertion section and is provided such that the direction of a visual field is displaceable.

8. An endoscope comprising:
an imaging unit at a tip end portion of an insertion section, wherein the imaging unit has
a board on which the image pickup device is installed,
an assembly section which is assembled on the board, and
a cable through which a signal is input to and output from the board,
wherein the assembly section and the cable are assembled on the board using a plurality of types of conductive connection materials which have different melting points.

9. The endoscope of claim 8,
wherein the imaging unit has a ground bar which is connected to a ground of the board and another assembling component as the assembly section, and
wherein the another assembling component and the ground bar are assembled on the board using a first conductive connection material, and the cable is assembled on the board using a second conductive connection material which has a lower melting point than that of the first conductive connection material.

10. The endoscope of claim 8,
wherein the imaging unit has a ground bar which is connected to a ground of the board and another assembling component as the assembly sections, and
wherein the another assembling component and a core line of the cable are assembled on the board using a first conductive connection material, and the ground bar and a shield part of the cable are assembled on the board using a second conductive connection material which has a lower melting point than that of the first conductive connection material.

11. A method of manufacturing an endoscope which includes an imaging unit at a tip end portion of an insertion section, the imaging unit having a board on which the image pickup device is installed, a cable through which a signal is input to and output from the board, and a ground bar which is provided so as to protrude from the surface of the board and is connected to a ground of the board, the method comprising:
assembling an assembly section and the cable to be assembled on the board on the board using a plurality of types of conductive connection materials which have different melting points, in a distal portion of the cable on the imaging unit side, when a shield part is connected to the ground of the board through the ground bar and a core line is directly assembled on the board.

12. The method of manufacturing an endoscope of claim 11, further comprising:
assembling the ground bar and another assembling component on the board using a first conductive connection material, and the cable on the board using a second conductive connection material which has a lower melting point than that of the first conductive connection material.

13. The method of manufacturing an endoscope of claim 11, still further comprising:
assembling the core line of the cable and the another assembling component on the board using a first conductive connection material, and the ground bar and the shield part of the cable on the board using a second conductive connection material which has a lower melting point than that of the first conductive connection material.

14. An endoscope comprising:
an imaging unit at a tip end portion of an insertion section, wherein the imaging unit has
a board on which the image pickup device is installed,
a cable through which a signal is input to and output from the board, and
a raising section which is provided so as to protrude from the surface of the board and is disposed on a ground of the board, and
wherein in a distal portion of the cable on the imaging unit side, a shield part is connected to the ground of the board in a state of being raised by the raising section and a core line is directly assembled on the board.
